(19) United States
(12) Patent Application Publication
(10) Pub. No.: US 2009/0226952 A1
(43) Pub. Date: Sep. 10, 2009

(54) SCREENING METHOD FOR COMPOUNDS WITH ANAESTHETIC PROPERTY

(75) Inventors: Susan Adele Greenfield, Oxfordshire (GB); Toby Francis Tristram Collins, Hampshire (GB)

Correspondence Address: SALIWANCHIK LLOYD & SALIWANCHIK;A PROFESSIONAL ASSOCIATION, PO Box 142950, GAINESVILLE, FL, 32614, US

(73) Assignee: Isis Innovation Limited, Oxford, Oxfordshire (GB)

(21) Appl. No.: 11/909,005

(22) PCT Filed: Mar. 17, 2006
(86) PCT No.: PCT/GB2006/050058

§ 371 Date: Nov. 18, 2008

(30) Foreign Application Priority Data
Mar. 18, 2005 (GB) 0505480.4

(51) Int. Cl. C12Q 1/02

(52) U.S. Cl. 435/29

(57) ABSTRACT

There is provided an in vitro screening method for compounds with anaesthetic property, which can distinguish anaesthetics from analgesics and relies on observing the ability of compounds to prolong the duration of transient working neuronal assemblies as may be generated, for example, in in vitro hippocampal slices and observed by optical imaging employing a voltage sensitive fluorescent dye.

The present invention relates to a convenient in vitro method for screening compounds for anaesthetic property. More particularly, it provides a method of identifying anaesthetics, and distinguishing anaesthetics from other drugs such as analgesics which have diverse inhibitory synaptic actions, relying on observing the effects of compounds on collective synchronised neuronal activity equating with transient working neuronal assemblies. Suitable such neuronal assemblies may be activated and observed by known electro-physiological techniques in in vitro brain slices, in particular, for example, in vitro hippocampal slices.

### BACKGROUND OF THE INVENTION

Anaesthetics and analgesics have long been known to have diverse synaptic actions that nonetheless have a common net inhibitory action on neuronal discharge. However, it has remained puzzling how these two classes of compound have significantly different net functions, one blocking pain and the other consciousness. Moreover, little has previously been known, beyond the isolated synapse, of the larger scale mechanisms that must be targeted specifically by anaesthetics to cause the defining loss of consciousness. The present invention stems from the finding that the temporal dynamics of transient working neuronal assemblies in the brain are modified selectively by anaesthetics but not by analgesics, irrespective of their effects at the synaptic level. More particularly, anaesthetics with entirely different synaptic actions in GABAergic systems have been shown to prolong the duration of transient working neuronal assemblies generated in in vitro hippocampal slices by paired pulse stimuli along the Schaffer Collaterals. This finding was made possible by application of an optical imaging technique using voltage sensitive fluorescent dyes that offers both a high (2 ms) time resolution combined with the requisite spatial resolution to observe neural activity across transient excited neuronal assemblies. This optical imaging technique is further described in Mann et al. J. Neuropharm. (2005) 48, 118-133, Grinvald et al., J. Neurosci. (1994) 14, 25645-25683 and in the example below.

As also detailed further in the exemplification below, the two anaesthetics, propofol and thiopental, whilst being chemically distinct and possessing different synaptic actions, both exemplify anaesthetics found by the inventors to prolong the duration of active neuronal assemblies generated in hippocampal slices by paired pulsing as described above. Rather than as under control conditions returning very quickly to baseline after the second stimulating pulse (within 60 ms of the peak activation), stimulated neuronal assemblies in the presence of propofol or thiopental remained in an activated state for an extended period (maintenance of activation of working neuronal assemblies was observed for up to 500 ms following peak activation arising from the second stimulating pulse). In contrast, the analgesics morphine and gabapentin, which also act at GABA receptors in the hippocampus, have no such effect. It is thus now envisaged that observation of the effect of compounds on neural activity across stimulated working neuronal assemblies can be of benefit for in vitro primary screening of compounds for anaesthetic property.

### SUMMARY OF THE INVENTION

In one aspect, the present invention thus provides a method of screening a compound for anaesthetic property, which comprises:
(i) stimulating in vitro transient activation of a working neuronal assembly, said transient activation being capable of prolongation in the presence of an anaesthetic and
(ii) determining whether said compound will cause such prolongation indicative of anaesthetic property.

Typically, as indicated above, such prolongation will be beyond 60 ms from peak activation following completion of the stimulation regime, e.g. following peak activation after completion of a paired-pulse electrical stimulation regime. Typically, in the presence of a compound with anaesthetic property, activation of the working neuronal assembly will be maintained significantly above base level for a substantial period beyond 60 ms from the stated base time point. Thus, for example, maintenance of activation may be observable at 100 ms, 150 ms, 200 ms, 250 ms, 300 ms, 350 ms, 400 ms, 450 ms, 500 ms from peak activation following completion of the stimulation regime. Maintenance of activation may be observable at far longer time points as further discussed below.

Any test compound thus identified in vitro as prolonging activation of a stimulated working neuronal assembly may be subsequently tested in vivo for anaesthetic property in a conventional manner.

A compound with “anaesthetic property” as thus identified will generally correspond to a compound having the clinical effect of an anaesthetic, i.e. capable of causing loss of consciousness. However, it will be understood that compounds identified in accordance with the invention may have clinical use other than purely as an anaesthetic.

By “working neuronal assembly” will be understood any transiently activated network of neurons observable as collective synchronised neuronal activity by an optical-imaging technique as described above and which is subject to more sustained activation in the presence of a known anaesthetic. As indicated above, it is well known how to activate such neuronal working assemblies in in vitro brain slices such as in vitro hippocampal slices. Transiently activated working neuronal assemblies may be tested for use in a method of the invention for example using any of the known barbiturate anaesthetics, e.g. thiopental (5-ethyl-5-(1-methylbutyl)-2-thiobarbituricacid), or any other anaesthetic recognised to affect GABAergic systems at the synaptic level in hippocampus, including for example propofol (2,6-diisopropylphenol). By use of such a working neuronal assembly in accordance with the invention further such anaesthetics having functionally equivalent effect in the hippocampus may, for example, be identified.

Compounds may be tested at more than one concentration in such a method thereby enabling quantification of anaesthetic property or determination of a minimum concentration for anaesthetic property. Interestingly, some compounds are known which change from analgesics at low concentration to anaesthetics at higher concentration, e.g. ketamine. It is envisaged that such compounds can be identified by applying a method of the invention with varying concentrations of the compound to be tested.

The invention is illustrated below with reference to the following figures providing results of the above-noted studies using hippocampal slices treated with the analgesics morphine and gabapentin and with the anaesthetics thiopental and propofol.

### BRIEF DESCRIPTION OF THE FIGURES

FIG. 1a: The equipment used showing arrangement of stimulating electrode (1), field potential recording electrode (2), halogen lamp (3) providing excitory wavelengths for the dye through a wavelength filter (4) and epi-fluorescent light pathway through a macroscope (5). FIG. 1b: calculation of time values from the maximum values (t₁ and t₃) of peaks 1 and 2, and their corresponding times for half-peak values (t₂ and t₄).

FIG. 2: Mean peak values from field potential recordings from in vitro rat hippocampal slices subjected to paired-pulse electrical stimulation of the Schaffer Collaterals in region CA3, under control and different treatment conditions (a) gabapentin (100 μM); (b) morphine (20 μM); (c) propofol (20 μM); (d) thiopental (100 μM). Control conditions, filled boxes; treatment conditions, open boxes. P₁, pulse 1; P₂, pulse 2. Error bars±SEM.

FIG. 3: Results of optical imaging using a voltage sensitive dye to highlight the activity of a population of neurons in response to paired-pulse electrical stimulation applied to the Schaffer Collaterals in rat hippocampal slices. Population activity recorded under control conditions in artificial cerebrospinal fluid (ACSF) was not substantially altered by the two analgesics, gabapentin and morphine. However, the two anaesthetic compounds tested, propofol and thiopental, caused prolonged activation following the second stimulating pulse observed at up to 500 ms (arrow).

FIG. 4: Differences between (a) electrophysiological and (b) optical imaging recording techniques in detecting the time course of activation of neuronal populations following peak 2 of a paired-pulse depolarisation paradigm.

### DETAILED DESCRIPTION

As evident from above, central to the invention is stimulating working neuronal assemblies which are subject to more sustained activation in the presence of an anaesthetic. Most conveniently, this may be achieved by stimulating a working neuronal assembly in an in vitro brain slice. While hippocampal slices were used for the exemplification, it is envisaged that any brain region may be employed which is associated with consciousness. Therefore, it is also envisaged that cortex slices might be alternatively employed. However, any form of working neuronal assembly may be employed provided it exhibits the required susceptibility to anaesthetics. This may be tested using known anaesthetics as discussed above.

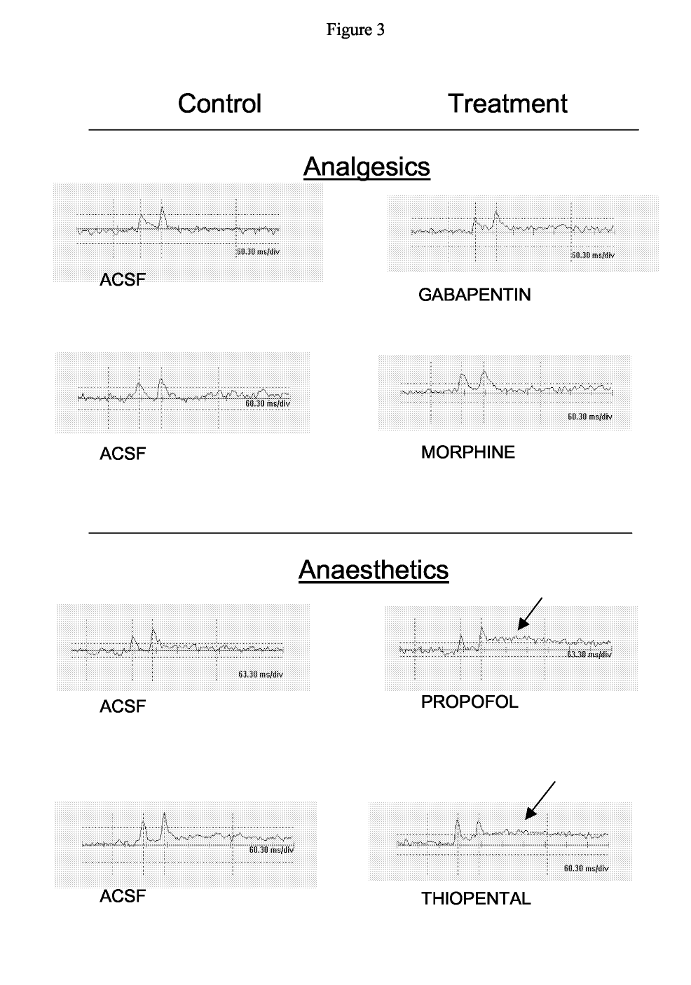

Stimulation of a working neuronal assembly may be achieved by any of the conventional techniques known for activating neurons. Such techniques include both chemical and electrical techniques. Preferably, however, electrical stimulation will be employed in the form of electrical pulses. Especially preferred is use of paired electrical pulses separated by a short interval, e.g. about 40 to 60 ms. As indicated above and described in the example below, in a preferred embodiment of the invention such paired pulses are applied to the Schaffer Collaterals of hippocampal slices in vitro. Where such electrical stimulation is employed, it is the ability of a compound to prolong the duration of the generated working neuronal assembly after peak activation following the end of the applied pulses which is of interest as indicative of anaesthetic property (see FIG. 3). In some instances, a larger train of electrical pulses may be employed, e.g. 3 or 4 such pulses in close succession, e.g. with a pulse interval as indicated above.

Stimulation of a transient working neuronal assembly, e.g. by application of paired electrical pulses in a hippocampal slice as described above, must be coupled with observation of collective synchronised neural activity in the presence of the compound to be tested. As already discussed above, this may be desirably achieved by application of optical imaging together with a voltage sensitive fluorescent dye, e.g. Di-4-ANEPPs. Thus, for example, where a brain slice is employed it may be submersed in a voltage-sensitive dye solution prior to stimulation.

Preferably, the compound to be tested will be applied in a perfusate in a closed circulating perfusion system. More than one concentration of the compound may be tested with washing between concentration changes. In this way, compounds may be identified which only display anaesthetic property above a certain critical concentration and anaesthetic property may be quantified. Alternatively, different concentrations of the same compound to be tested may be contacted with separate, equivalent activated working neuronal assemblies, e.g. within identically stimulated hippocampal slices.

Any significant prolongation of collective neural activity beyond that observed with control conditions will be of interest. Thus, typically by “significant prolongation” will be understood maintenance of activation significantly above base level beyond 60 ms from peak activation following completion of the stimulation regime, e.g. following peak activation associated with the second pulse of a paired-pulse electrical stimulation regime. As indicated above, anaesthetic property can be anticipated to correlate with maintenance of activation of a stimulated working neuronal assembly for considerably longer than 60 ms, e.g. maintenance of activation may be observable at about 100 ms, about 150 ms, about 200 ms, about 250 ms, about 300 ms, about 350 ms, about 400 ms, about 450 ms, about 500 ms from peak activation following the end of the stimulation regime.

In some instances, it may be chosen to observe the level of activation for longer than about 500 ms, e.g. in the presence of a compound with anaesthetic property activation may still be observable at time points well beyond 500 ms, e.g. at any 50 to 100 ms interval up to about 1 s, up to about 1.5 s, up to about 2 s, up to about 2.5 s, up to about 3 s, up to about 4 s, up to about 5 s or even longer. However, where optical imaging is employed with a voltage sensitive dye, the time period selected for data collection will necessarily be below times at which there is possibility of photo-bleaching and photo-toxicity. Hence, generally it will be found convenient to monitor activation of the stimulated neuronal assembly for no longer than about 1 s. Moreover, as shown by the exemplification, where a working neuronal assembly is generated in a hippocampal slice, an observation period of 500 ms or less from peak activation following the end of the stimulation regime will suffice to distinguish an anaesthetic which acts in the hippocampus from an analgesic which also acts in that brain region. When employing such a hippocampal slice test system, prolongation of activation of a stimulated working neuronal assembly may be judged with reference to the time course of reduction in activation observed in the presence of one or more analgesics, e.g. gabapentin and/or morphine. In this case, any activation above that observed with analgesic at about 60 to 70 ms from peak activation following the end of stimulation will be judged significant.

The level of activation of an appropriate stimulated neuronal population may be observed at a single time point following the end of stimulation which is known to correlate with possession of desired anaesthetic property. However, generally it will be chosen to monitor the level of activation for a period following the end of stimulation. Thus, for example, compounds of interest may be selected on the basis of maintenance of activation of the stimulated working neuronal assembly being observed for at least up to about 100 ms, about 200 ms, about 250 ms, about 300 ms, about 350 ms, about 400 ms, about 450 ms, about 500 ms or longer from peak activation after the end of the stimulation regime. Preferably, compounds may be selected for example on the basis of observation of maintenance of activation from 100 ms to about 300 ms, about 400 ms, about 450 ms, about 500 ms or longer, most preferably 100 to 500 ms, following peak activation after completion of the stimulation regime.

The following Example illustrates the invention.

### EXAMPLE

As indicated above, the effects of two analgesics (morphine and gabapentin) and two anaesthetics (thiopental and propofol) were observed on transient working neuronal assemblies in in vitro hippocampal slices using an optical imaging technique employing a voltage sensitive fluorescent dye. The selected drugs were all known to affect GABAergic systems at the synaptic level in the hippocampus, but show a wide diversity of chemical structure and mechanisms of synaptic action. The analgesic morphine suppresses GABA receptor-induced inhibition in hippocampal pyramidal cells^{4, 5}, and disrupts gamma oscillations⁶ whilst the less familiar antagonist of neuropathic pain, gabapentin, has been implicated as a GAB_{AB}(1a, 2) receptor agonist that activates postsynaptic K⁺ currents and leads to the inhibition of postsynaptic Ca2+ currents in CA1 hippocampal pyramidal cells⁷. The sedative-hypnotic anaesthetic propofol enhances the inhibitory inputs mediated by GABA_{A} receptors^{8, 9}, in turn activated by NMDA currents¹⁰, by sodium channels¹¹, by voltage-gated calcium channels¹², by the inhibition of hyperpolarization-activated inward current (IH)¹³ and by interactions with GABA_{B} receptors¹⁴. In contrast, the barbiturate anaesthetic thiopental operates via GABA_{A} receptors¹⁵ and in so doing disrupts the synchrony of gamma and beta oscillations, thereby preventing excitatory synaptic potentiation in the hippocampus^{16, 17}.

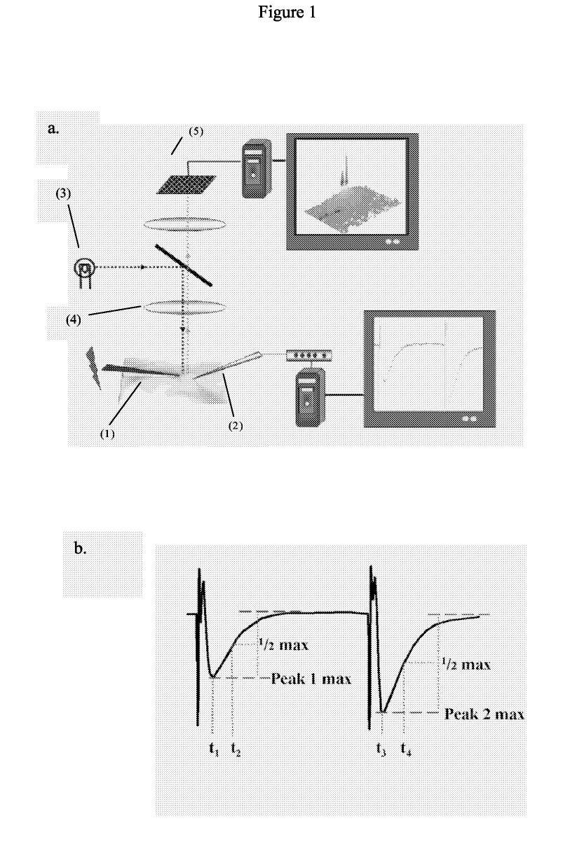

In the study, collective synchronised neuronal activity, i.e. working neuronal assemblies³, were recorded while monitoring in parallel the underlying synaptic electrical activity (FIG. 1a). Accordingly, the use of the voltage sensitive dye, Di-4-ANEPPS, was combined with standard electrophysiological field potential recordings to measure the excitatory post-synaptic potentials (EPSPs). Under control conditions in physiological saline, electrical stimuli in a paired-pulse paradigm¹⁸ were applied to the Schaffer Collaterals of in vitro hippocampal slices. These stimuli elicited spreading depolarisations that formed a transient neuronal assembly, which formed at the point of stimulation and spread to the CA1 region. After a brief period of activity, the excited region returned to rest.

### Methods

Following terminal anaesthesia using halothane, 400 μm transverse hippocampal slices from 2 week Wistar rats were obtained and left to rest for one hour in a high humidity chamber with oxygenated artificial cerebrospinal fluid (ACSF) (in mM: 124 NaCl, 22 NaHCO₃, 10 glucose, 5 KCl, 2 CaCl₂, 1.25 MgSO₄, 1.25 NaH₂PO₄) before being submersed in dye solution (4% dye stock solution (in mM: 0.2 mM Di-4-ANEPPS (Molecular Probes, Oregon, USA) in 2.7% ethanol, 0.13% Cremaphor EL), 48% fetal bovine serum and 24% ACSF and 24% ACSF cellulose) for 25 minutes followed by a further one hour rest in the humidity chamber with oxygenated ACSF.

Electrical stimulation (paired pulse 8-10V, 60 ms interval, 0.1 ms duration) was generated by an isolated stimulator (Digitimer Ltd, UK) via a bipolar electrode (FHC, Maine, USA) and positioned just above the CA3 region. In this way, paired pulses were applied along the Shaffer Collaterals so as to elicit a spreading depolarising wave that formed from the point of stimulation and moved along the pathway towards the CA1 region. A halogen lamp provided the excitatory wavelengths for the dye (through a λ=530±10 nm filter) while the emission was captured on a 2 mm×3 mm CCD sensor (MiCAM01, BrainVision, Tokyo, Japan) through an absorption filter (λ=590 nm). Optical recordings were acquired at a rate of 0.7 ms frame⁻¹ for 682 frames. 16 optical recordings were averaged for each experimental condition to reduce the effects of dye bleaching and increase the signal-to-noise ratio. Analysis included ΔF/F calculations and a 5×5×3 Gaussian matrix filter (BrainVision Analysis software and Igor Pro 5, Wavemetrics, USA). As indicated above, such optical imaging was combined with standard electrophysiological field potential recordings to measure the excitory post synaptic potentials (EPSPs). All chemicals were from Sigma except gabapentin (Tocris) and propofol (Zeneca). All statistical comparisons were performed using student t-test of paired samples, n=6; 95% confidence interval.

### Results

As indicated above, under control conditions in physiological saline, paired electrical stimuli elicited a spreading depolarising wave towards the CA1 region and, after a brief period of activity, the excited region soon returned to rest (FIG. 1a). Paired-pulse facilitation was observed in the control response in both the optical signal and the EPSP trace.

In the presence of morphine (10-100 μM), the depolarising wave originated at the stimulating electrode, spread along the Schaffer Collaterals with similar activation and inactivation characteristics to the controls, and the neurons returned to a resting state within 60 ms of the peak activation arising from the second pulse. There were significant increases in the EPSP responses between control and treatment conditions in both peak 1 and peak 2 values (peak 1, p=0.0058; peak 2, p=0.0088). Paired-pulse facilitation was recorded from the EPSPs in controls and treatments but there was no significant enhancement of facilitation with application of morphine (p=0.5371).

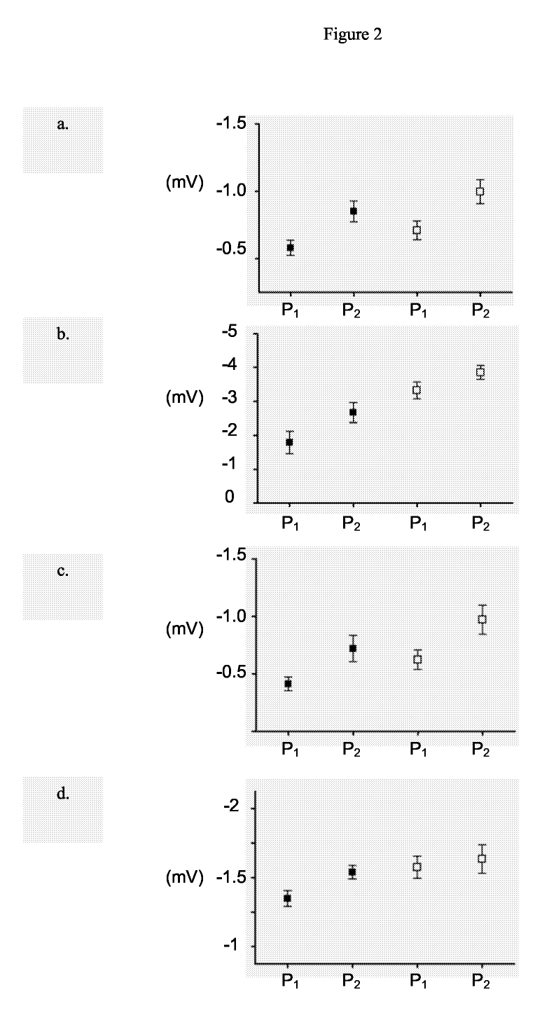

When gabapentin was applied to slices (100-150 μM), the EPSPs were not significantly changed from controls (FIG. 2a) although there was some reduction in the second peak (peak 1, p=01806; peak 2, p=0.0748). The paired pulse paradigm did not cause facilitation in the EPSP recordings and there was no significant difference between control and treatment conditions. As with morphine, the neurons returned to a resting state within 60 ms after peak activation following the second stimulating pulse (p=0.4864).

When the barbiturate anaesthetic thiopental was given (80-150 μM), the excitatory spread from the stimulation source was still evident, and this followed the same pathway as the controls. The EPSP signal was significantly reduced from control values (peak 1, p=0.0024; peak 2, p=0.0012). Paired-pulse facilitation was evident under control conditions but was abolished following addition of thiopental (FIG. 2d). Significantly, the optical imaging showed that rather than returning to the baseline level within 60 ms of the peak activation after the second stimulating pulse, the neurons remained in an activated state for up to 500 ms, before eventually returning to resting baseline level. This response was removed after washing the slices in physiological saline.

A similar response was seen by optical imaging after treating slices with 10-50 μM propofol. Importantly, as with thiopental, the activation after the second pulse did not return to baseline even after up to 500 ms. EPSP recordings showed that both peaks were significantly reduced from their control values (peak 1, p=0.0127; peak 2, p=0.0121). Paired-pulse facilitation was evident in both control and treatment conditions, but the ratios did not significantly differ between controls and treatments.

In more detail, the optical ΔF/F recordings revealed a divergent picture. Gabapentin treatment versus control showed a significant difference in peak values (peak 1, p=0.0003; peak 2, p=0.0064). Paired-pulse facilitation was recorded under both control and gabapentin conditions, with significant difference between the two treatments (p=0.0129).

The ΔF/F recordings of morphine treatment, like those from the EPSP signal, showed a slight increase in the spread and intensity of neuronal activation but were not significantly different from controls (peak 1, p=0.1098; peak 2, p=0.1485). Paired-pulse facilitation was evident under both control and treatment conditions but no significant enhancement of facilitation was evident after morphine addition (p=0.5599).

Propofol treatment values for peak 1 did not differ significantly from controls (p=0.6867) but there was significant reduction of peak 2 (p=0.031). Paired-pulse facilitation was evident for both control and treatment conditions and the ratios did not significantly change.

Control and thiopental treatment values did not differ significantly for peak 1 (p=0.2585) but there was significant reduction in the value of peak 2 (p=0.0111). The ΔF/F recordings highlighted the presence of paired-pulse facilitation under control conditions, but this was abolished in the presence of thiopental. From both the EPSP and ΔF/F data, measurements were made of the time from peak activity to a value of half peak activity, from both peak 1 and peak 2. In all four treatments, there was no significant difference in the peak 1 (t₁−t₂) values from controls.

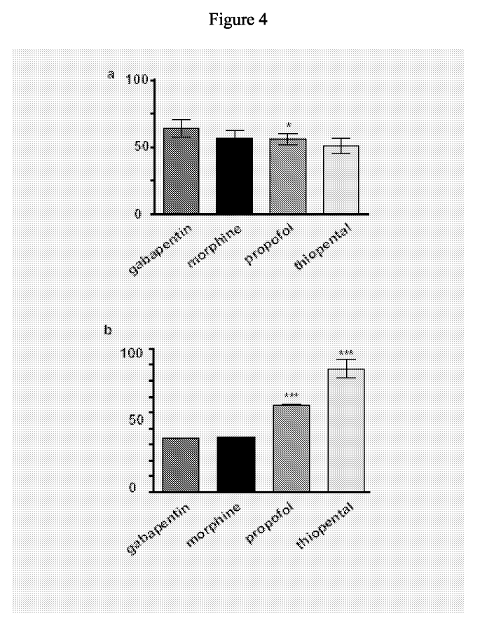

Following gabapentin and morphine addition, both EPSP and ΔF/F recordings of peak 2 activation of neurons showed return to a resting state within 60 ms of the peak activation and there was no significant difference between treatments and controls. Surprisingly however, following the addition of propofol, the EPSP data for peak 2 (t₃−t₄) values had a low statistical difference (p=0.0360) from controls but the ΔF/F recordings showed very highly significant differences (p=0.001). Thiopental caused no significant change in EPSP recorded data but the ΔF/F recordings, for peak 2 (t₃−t₄), were highly significant from controls (p=0.006) (see FIG. 4)

### CONCLUSION

The measurement of neuronal population activity 60 ms after the maximum value of the second stimulating pulse reveals the nature of the drug being tested. Anaesthetic compounds show a prolonged activation, detectable beyond 60 ms after the maximum value of the second stimulating peak. However, under control conditions and in the presence of analgesic compounds no such activity was measured; the neurones returned to a resting state before the critical 60 ms or longer time frame.

In the exemplification, it was shown that at time periods up to 500 ms from the peak activation corresponding to the second stimulating pulse activation of the stimulated working neuronal assembly could still be observed in the presence of an anaesthetic. However, longer time periods of observation are not ruled out as being suitable and might be coupled with use of other technologies for determining collective neuronal activity, for example functional magnetic resonance imaging (fMRI) or electroencephalogram (EEG)

The validity has been discussed previously of the value of the paired pulse paradigm¹⁸ as well as the optical imaging technique, used here in in vitro hippocampus². Clearly, and perhaps not surprisingly, additional information was gained from the optical imaging that could not have been apparent from the field potential recordings. However, one finding was completely unexpected and dependent upon the visualisation of large scale neuronal coordination: the significant prolongation of assembly duration by anaesthetics alone. This effect cannot be attributed to the those drugs washing out more slowly than others as, in each case, the test compound was added to a closed circulating perfusion system and was, therefore, constantly present. Moreover, increases in synaptic action can also be discounted as the critical factor as morphine showed an increase in excitatory action, but did not cause a persistent increase in synaptic activity following the stimulating pulses. Conversely, inhibitory synaptic action cannot be considered relevant as gabapentin shares a depressant synaptic action with the anaesthetic compounds but does not cause the prolonged activation response shown by anaesthetics. Propofol and thiopental, which are chemically distinct and possess different synaptic actions are, however, comparable functionally in that they are both anaesthetics. Hence, it is postulated that beyond isolated and individual chemical action and synaptic activity, neuronal assembly dynamics are linked to loss, and hence the generation, of consciousness.

### REFERENCES

1. Mann, E. O., Tominaga, T., Ichikawa M. & Greenfield, S. A. (2005). Cholinergic modulation of the spatiotemporal pattern of hippocampal activity in vitro. J. Neuropharm. 48:118-133.

2. Grinvald, A., Lieke, E. E., Frostig, R. D., & Hildesheim, R. (1994). Cortical point-spread function and long-range lateral interactions revealed by real-time optical imaging of Macaque monkey primary visual cortex. J Neurosci., 14: 2545-25683.

3. Greenfield, S. A. The Private Life of the Brain. Penguin. London. (2000)

4. Corigall, W. A. & Linseman, M. A. A specific affect of morphine on evoked activity in the rat hippocampal slice. Brain Res. 192 (1), 227-238 (1980)

5. Swearengen E. & Chavkin, C. Comparison of opioid and GABA receptor control of excitability and membrane conductance in hippocampal CA1 pyramidal cells in rat. Neuropharmacology, 28 (7), 689-697 (1989)

6. Whittington, M. A., Traub, R. D., Faulkner, H. J., Jefferys, J. G. R. & Chettiar, K. Morphine disrupts long-range synchrony of gamma oscillations in hippocampal slices. Proc. Natl. Acad. Sci. USA. 95, 5807-5811, (1998)

7. Bertrand S., Nouel, D., Morin, F., Nagy, F. & Lacaille, J. C. Gabapentin actions on Kir3 currents and N-type Ca²⁺ channels via GABAB receptors in hippocampal pyramidal cells. Synapse, 50, 95-109 (2003)

8. Bieda, M. C. & MacIver, M. B. Major role for tonic GABA_{A} conductances in anaesthetic suppression of intrinsic neuronal excitability. J. Neurophysiol. 92, 1658-1667 (2004).

9. Peduto, V. A., Concas, A., Snatoro, G., Biggio, G & Gassa, G. L. Biochemical and electrophysiological evidence that propofol enhances GABAergic transmission in the rat brain. Anaesthesiology 75, 1000-1009 (1991)

10. Orser, B. A., Bertlik, M., Wang, L. Y. & MacDonald, J. F. Inhibition by propofol (2,6 di-isopropylphenol) of the N-methyl-D-aspartate subtype of glutamate receptor in cultured rat hippocampal neurones. Br. J. Pharmacol. 116, 1761-1768 (1995)

11. Rehberg, B. & Duch, D. S. Suppression of central nervous system sodium channels by propofol. Anaesthesiology 91, 512-520 (1999)

12. Inoue, Y., Shibuya, I. Kabashima, N., Noguchi, J., Harayama, N., Ueta, Y., Sata, T., Shigematsu, A. & Yamashita, H. The mechanism of inhibitory actions of propofol on rat supraoptic neurones. Anaesthesiaology 91, 167-178

13. Funahashi, M., Higuchi, H., Miyawaki, T., Shimida, S. & Matsuo, R. Propofol suppresses a hyperpolarization-activated inward current in rat hippocampal CA1 neurons. Neurosci. Lett. 311, 177-180 (2001).

14. Scwhieler, L., Delbro, D. S., Engberg, G. & Erhardt, S. The anaesthetic agent propofol interacts with GABAB-receptors: an electrophysiological study in rat. Life Sciences. 72, 2793-2801 (2003).

15. Hirota, K., Roth, S. H., Fujimura, J, Masuda, A. & Ito, Y. GABAergic mechanisms in the actions of general anaesthesics. Toxicol. Letters. 100-101, 203-207 (1998).

16. Faulkner, H. J., Traub, R. D. & Whittington, M. A. Anaesthetic/amnesic agents disrupt beta frequency oscillations associated with potentiation of excitatory synaptic potentials in the rat hippocampal slice. Br. J. Pharmacol. 128, 1813-1825 (1999).

17. Faulkner, H. J., Traub, R. D. & Whittington, M. A. Disruption of synchronous gamma oscillations in the rat hippocampal slice: a common mechanism of anaesthetic drug action. Br. J. Pharmacol. 125, 483-492 (1998).

18. Davies, C. H., Davies, S. N. & Collingridge, G. L. Paired-pulse depression of monosynaptic GABA-mediated inhibitory postsynaptic responses in rat hippocampus. J. Physiol. 424, 513-531 (1990).

1-11. (canceled)

12. A method for screening a compound for anesthetic property, which comprises:
(i) stimulating in vitro transient activation of a working neuronal assembly, said transient activation being capable of prolongation in the presence of an anesthetic, and
(ii) determining whether said compound will cause such prolongation indicative of anesthetic property.

13. The method according to claim 12, wherein said working neuronal assembly is activated within an in vitro brain slice.

14. The method according to claim 13, wherein said brain slice is a hippocampal slice.

15. The method according to claim 12, wherein said stimulating is by means of pulsed electrical stimuli or paired pulse electrical stimuli, and said determining is carried out after the final pulse.

16. The method according to claim 15, wherein paired-pulse electrical stimuli are applied along the Schaffer Collaterals in a hippocampal slice.

17. The method according to claim 12, wherein determining is by means of optical-imaging of fluorescence of a voltage-sensitive fluorescent dye.

18. The method according to claim 12, wherein anesthetic property is correlated with prolongation of during of the working neuronal assembly activation beyond 60 ms from peak activation following the end of the stimulation regime.

19. The method according to claim 18, wherein anesthetic property is correlated with prolongation of duration of the working neuronal assembly activation for at least 100 ms to about 500 ms or longer from peak activation following the end of the stimulation regime.

20. The method according to claim 19, wherein a hippocampal slice is employed, step (i) is carried in accordance with claim 16 and said determining is by optical imaging in accordance with claim 17.

21. The method according to claim 12, wherein steps (i) and (ii) are repeated with different concentrations of the compound to be tested.

22. The method according to claim 21, wherein the test compound is an analgesic which exhibits anesthetic property at above a critical concentration.

23. The method according to claim 13, wherein said stimulating is by means of pulsed electrical stimuli or paired pulse electrical stimuli, and said determining is carried out after the final pulse.

24. The method according to claim 14, wherein said stimulating is by means of pulsed electrical stimuli or paired pulse electrical stimuli, and said determining is carried out after the final pulse.

25. The method according to claim 13, wherein said in vitro brain slice is a hippocampal slice and paired-pulse electrical stimuli are applied along the Schaffer Collaterals in said hippocampal slice.
